(19) United States
(12) Patent Application Publication
(10) Pub. No.: US 2013/0150386 A1
(43) Pub. Date: Jun. 13, 2013

(54) METHODS FOR THE TREATMENT OF LUNG CANCER

(71) Applicant: Syndax Pharmaceuticals, Inc., Waltham, MA (US)

(72) Inventors: Robert Goodenow, San Clemente, CA (US); Peter Ordentlich, San Diego, CA (US)

(73) Assignee: SYNDAX PHARMACEUTICALS, INC., Waltham, MA (US)

(21) Appl. No.: 13/708,856

(22) Filed: Dec. 7, 2012

Related U.S. Application Data
(60) Provisional application No. 61/569,135, filed on Dec. 9, 2011.

(51) Int. Cl. A61K 45/06; A61K 31/517; A61K 31/4406

(52) U.S. Cl. CPC A61K 45/06; A61K 31/4406; A61K 31/517
USPC 514/266.4; 514/357

(57) ABSTRACT

Described herein are methods for the treatment of lung cancer in a subject. In particular, methods are provided for the treatment of resistant lung cancer with a combination of entinostat and an EGFR inhibitor.

### CROSS REFERENCE

This application claims the benefit of U.S. Provisional Application No. 61/569,135, filed Dec. 9, 2011, which is incorporated herein by reference in its entirety.

### FIELD

The present invention relates to methods for the treatment of lung cancer based on the administration HDAC inhibitors and EGFR inhibitors.

### BACKGROUND

Cancer, tumors, tumor-related disorders, and neoplastic disease states are serious and often times life-threatening conditions. These diseases and disorders, which are characterized by rapidly-proliferating cell growth, continue to be the subject of research efforts directed toward the identification of therapeutic agents which are effective in the treatment thereof. Such agents prolong the survival of the patient, inhibit the rapidly-proliferating cell growth associated with the neoplasm, or effect a regression of the neoplasm.

Generally, surgery and radiation therapy are the first modalities considered for the treatment of cancer that is considered locally confined, and offer the best prognosis. Chemotherapy treatment of certain cancers typically results in disappointing survival rates but still offer a survival benefit. For example, in patients with lung cancer, EGFR inhibitor chemotherapy regimens, such as the use of erlotinib and gefitinib are employed. If patients fail to respond to an EGFR inhibitor treatment, additional conventional treatment, as currently employed, offers limited benefit.

While several EGFR inhibitors have been approved for the treatment of lung cancer, EGFR inhibitor therapy encounters limitations, such as side-effects resulting from its use. Of greater concern, is the growing view that, while utilization of EGFR inhibitors for the treatment of tumors may initially shrink the size of the tumor, the tumor may eventually enlarge in size, indicating, among other things, the development of resistance. Erlotinib, a widely used EGFR inhibitor, may be representative of the types of therapeutic agents being used for cancer treatment in that its use has an effect on cancer, but because of other factors, which are not entirely known, the tumor develops resistance and progresses.

HDAC inhibitors are an emerging class of therapeutic agents that promote differentiation and apoptosis in hematologic and solid malignancies through chromatin remodeling and gene expression regulation. Several HDAC inhibitors have been identified including benzamides (entinostat), short-chain fatty acids (i.e., Sodium phenylbutyrate); hydroxamic acids (i.e., suberoylanilide hydroxamic acid and thrichostatin A); cyclic tetrapeptides containing a 2-amino-8-oxo-9,10-epoxy-decanoyl moiety (i.e., trapoxin A) and cyclic peptides without the 2-amino-8-oxo-9,10-epoxy-decanoyl moiety (i.e., FK228). Entinostat is a benzamide HDAC inhibitor undergoing clinical investigation in multiple types of solid tumors and hematologic cancers. Entinostat is rapidly absorbed and has a half-life of about 100 hours and, importantly, changes in histone acetylation persist for several weeks following the administration of entinostat.

What is needed, therefore, are compositions and/or methods of treatment for cancer which take advantage of the synergy found in a therapeutic combination that could increase the effectiveness of the agents and reduce and/or eliminate the side effects typically associated with conventional treatments.

### SUMMARY OF THE INVENTION

Another embodiment provides a method of treating cancer in an EGFR inhibitor-naïve patient progressed on prior therapy, wherein the method comprises: (1) determining the E-cadherin expression level in the patient; (2) selecting the patient exhibiting a high E-cadherin expression level scored as +3; and (3) administering to the patient a combination comprising entinostat and an EGFR inhibitor.

Another embodiment provides the method wherein high E-cadherin expression levels are determined by ELISA, immunohistochemistry, immunocytochemistry or determination of E-cadherin methylation levels. Another embodiment provides the method wherein high E-cadherin expression levels are determined by immunohistochemistry. Another embodiment provides the method wherein the high E-cadherin expression levels are scored as +3 as determined by immunohistochemistry.

Another embodiment provides the method wherein the cancer is lung cancer. Another embodiment provides the method wherein the lung cancer is non-small cell lung cancer.

Another embodiment provides the method wherein the EGFR inhibitor administered in combination with entinostat is erlotinib.

Another embodiment provides the method wherein entinostat and the EGFR inhibitor are administered sequentially in either order or simultaneously. Another embodiment provides the method wherein entinostat and the EGFR inhibitor are administered simultaneously. Another embodiment provides the method wherein the EGFR inhibitor is administered first.

Another embodiment provides the method wherein the EGFR inhibitor is administered daily and the entinostat is administered periodically. Another embodiment provides the method wherein the EGFR inhibitor is administered daily and the entinostat is administered weekly.

another embodiment provides a method of treating cancer in an EGFR inhibitor-naïve patient progressed on prior therapy, wherein said patient exhibits high E-cadherin expression levels, the method comprising administering to the patient a combination comprising entinostat and an EGFR inhibitor.

One embodiment provides a kit for treating advanced non-small cell lung cancer comprising a combination of entinostat and an EGFR inhibitor and instructions for the administration of the dosage form.

Another embodiment provides a kit wherein the kit comprises one entinostat dosage form for every seven EGFR inhibitor dosage forms. Another embodiment provides a kit wherein the kit comprises two entinostat dosage forms for every 14 EGFR inhibitor dosage forms. Another embodiment provides a kit wherein the kit comprises 4 entinostat dosage forms and 28 EGFR inhibitor dosage forms. Another embodiment provides a kit wherein the EGFR inhibitor is erlotinib.

Another embodiment provides the method of treating cancer in an EGFR inhibitor-naïve patient progressed on prior therapy, wherein said patient exhibits high E-cadherin expression levels, wherein the method further comprises administering to the subject one or more additional therapies in addition to the combination of entinostat and the EGFR inhibitor. Another embodiment provides the method wherein the one or more therapies comprise one or more of radiation therapy, chemotherapy, high dose chemotherapy with stem cell transplant, and monoclonal antibody therapy. Another embodiment provides the method wherein radiation therapy comprises internal and/or external radiation therapy. Another embodiment provides the method wherein the chemotherapy comprises administering to the subject one or more of doxorubicin, cyclophosphamide, paclitaxel, lapatinib, capecitabine, trastuzumab, bevacizumab, gemcitabine, eribulin, or nab-paclitaxel. Another embodiment provides the method wherein the chemotherapy comprises administering to the subject one or more IGF-1R inhibitors. Another embodiment provides the method wherein the IGF-1R inhibitor is AEW541.

### INCORPORATION BY REFERENCE

All publications, patents, and patent applications described in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 provides a summary of the Phase II clinical trial;

FIG. 2 provides a summary of entinostat properties;

FIG. 3 provides an summary of the study design in the Phase II clinical trial;

FIG. 4 provides a summary of biomarkers and methods for analysis;

FIG. 5 provides a summary eligibility criteria for participation in the study;

FIG. 6 provides a summary of the patient population participating in the study described herein;

FIG. 7 provides a summary of biomarker status of the patient population participating in the study described herein;

FIG. 8 provides a summary of progression-free survival and overall survival observed during the Phase II clinical trial;

FIG. 9 provides a summary of adverse events observed during in the Phase II clinical trial;

FIG. 10 provides examples of E-cadherin protein level expression classification based on IHC analysis;

FIG. 11 provides an analysis of overall survival based upon E-cadherin levels;

FIG. 12 provides an analysis of progression-free survival based upon E-cadherin levels; and

FIG. 13 provides a summary of the outcome of the Phase II clinical trial.

### DETAILED DESCRIPTION

Provided herein are methods of treating cancer based on the administration of an HDAC inhibitor and an EGFR inhibitor. The methods may further include treatments wherein the combination is supplemented with one or more therapeutic agents or therapies.

To facilitate understanding of the disclosure set forth herein, a number of terms are defined below.

As used herein, “abnormal cell growth,” refers to cell growth that is independent of normal regulatory mechanisms (e.g., loss of contact inhibition), including the abnormal growth of normal cells and the growth of abnormal cells.

“Neoplasia” as described herein, is an abnormal, unregulated and disorganized proliferation of cells that is distinguished from normal cells by autonomous growth and somatic mutations. As neoplastic cells grow and divide they pass on their genetic mutations and proliferative characteristics to progeny cells. A neoplasm, or tumor, is an accumulation of neoplastic cells. In some embodiments, the neoplasm can be benign or malignant.

“Metastasis,” as used herein, refers to the dissemination of tumor cells via lymphatics or blood vessels. Metastasis also refers to the migration of tumor cells by direct extension through serous cavities, or subarachnoid or other spaces. Through the process of metastasis, tumor cell migration to other areas of the body establishes neoplasms in areas away from the site of initial appearance.

As discussed herein, “angiogenesis” is prominent in tumor formation and metastasis. Angiogenic factors have been found associated with several solid tumors such as rhabdomyosarcomas, retinoblastoma, Ewing sarcoma, neuroblastoma, and osteosarcoma. A tumor cannot expand without a blood supply to provide nutrients and remove cellular wastes. Tumors in which angiogenesis is important include solid tumors such as renal cell carcinoma, hepatocellular carcinoma, and benign tumors such as acoustic neuroma, and neurofibroma. Angiogenesis has been associated with blood-born tumors such as leukemias. It is believed that angiogenesis plays a role in the abnormalities in the bone marrow that give rise to leukemia. Prevention of angiogenesis could halt the growth of cancerous tumors and the resultant damage to the subject due to the presence of the tumor.

The term “subject” refers to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

The terms “treat,” “treating,” and “treatment” are meant to include alleviating or abrogating a disorder, disease, or condition; or one or more of the symptoms associated with the disorder, disease, or condition; or alleviating or eradicating the cause(s) of the disorder, disease, or condition itself.

The term “therapeutically effective amount” refers to the amount of a compound that, when administered, is sufficient to prevent development of, or alleviate to some extent, one or more of the symptoms of the disorder, disease, or condition being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The term “pharmaceutical composition” refers to a mixture of a compound disclosed herein with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

### Methods for the Treatment of Lung Cancer

One embodiment provides a method of treating cancer in an EGFR inhibitor-naïve patient progressed on prior therapy, wherein the method comprises: (1) determining the E-cadherin expression level in the patient; (2) selecting the patient exhibiting a high E-cadherin expression level scored as +3; and (3) administering to the patient a combination comprising entinostat and an EGFR inhibitor.

Another embodiment provides the method wherein the prior therapy was one prior chemotherapy.

Another embodiment provides the method wherein the prior therapy was two or more prior chemotherapies.

Another embodiment provides the method wherein high E-cadherin expression levels are determined by ELISA, immunohistochemistry, immunocytochemistry or determination of E-cadherin methylation levels. Another embodiment provides the method wherein high E-cadherin expression levels are determined by immunohistochemistry. Another embodiment provides the method wherein the high E-cadherin expression levels are scored as +3 as determined by immunohistochemistry.

Another embodiment provides the method wherein the cancer is lung cancer.

Another embodiment provides the method wherein the lung cancer is non-small cell lung cancer.

Another embodiment provides the method wherein the EGFR inhibitor administered in combination with entinostat is erlotinib.

Another embodiment provides the method wherein entinostat and the EGFR inhibitor are administered sequentially in either order or simultaneously. Another embodiment provides the method wherein entinostat and the EGFR inhibitor are administered simultaneously. Another embodiment provides the method wherein the EGFR inhibitor is administered first.

Another embodiment provides the method wherein the EGFR inhibitor is administered daily and the entinostat is administered periodically. Another embodiment provides the method wherein the EGFR inhibitor is administered daily and the entinostat is administered weekly.

Another embodiment provides a method of treating cancer in an EGFR inhibitor-naïve patient progressed on prior therapy, wherein said patient exhibits high E-cadherin expression levels, the method comprising administering to the patient a combination comprising entinostat and an EGFR inhibitor.

One embodiment provides a kit for treating advanced non-small cell lung cancer comprising a combination of entinostat and an EGFR inhibitor and instructions for the administration of the dosage form.

Another embodiment provides a kit wherein the kit comprises one entinostat dosage form for every seven EGFR inhibitor dosage forms.

Another embodiment provides a kit wherein the kit comprises two entinostat dosage forms for every 14 EGFR inhibitor dosage forms.

Another embodiment provides a kit wherein the kit comprises 4 entinostat dosage forms and 28 EGFR inhibitor dosage forms.

Another embodiment provides a kit wherein the EGFR inhibitor is erlotinib.

Another embodiment provides the method of treating cancer in an EGFR inhibitor-naïve patient progressed on prior therapy, wherein said patient exhibits high E-cadherin expression levels, wherein the method further comprises administering to the subject one or more additional therapies in addition to the combination of entinostat and the EGFR inhibitor. Another embodiment provides the method wherein the one or more therapies comprise one or more of radiation therapy, chemotherapy, high dose chemotherapy with stem cell transplant, and monoclonal antibody therapy. Another embodiment provides the method wherein radiation therapy comprises internal and/or external radiation therapy. Another embodiment provides the method wherein the chemotherapy comprises administering to the subject one or more of doxorubicin, cyclophosphamide, paclitaxel, lapatinib, capecitabine, trastuzumab, bevacizumab, gemcitabine, eribulin, or nab-paclitaxel. Another embodiment provides the method wherein the chemotherapy comprises administering to the subject one or more IGF-1R inhibitors. Another embodiment provides the method wherein the IGF-1R inhibitor is AEW541.

### Histone Deacetylase

The HDACs are a family including at least eighteen enzymes, grouped in three classes (Class I, II and III). Class I HDACs include, but are not limited to, HADCs 1, 2, 3, and 8. Class I HDACs can be found in the nucleus and are believed to be involved with transcriptional control repressors. Class II HDACs include, but are not limited to, HDACS 4, 5, 6, 7, and 9 and can be found in both the cytoplasm as well as the nucleus. Class III HDACs are believed to be NAD dependent proteins and include, but are not limited to, members of the Sirtuin family of proteins. Non-limiting examples of sirtuin proteins include SIRT1-7. As used herein, the term “selective HDAC” refers to an HDAC inhibitor that does not interact with all three HDAC classes.

### EGFR

In the last few years, knowledge about molecular mechanisms and cellular transformation in association with cancer behavior has increased. More interest has been generated since the development of specific targeted therapies against the processes involved in the carcinogenesis of many types of cancers. During the 1990s it was discovered that the EGFR played an important role in tumoral biology and behavior. EGFR stimulation activates intracellular signaling and cascades that influence cellular proliferation and mobilization, angiogenesis and other mechanisms. Normal cells are influenced by external factors, in tumor cells it was found that the activation of cell proliferation mediated by this receptor would no longer need external stimuli, but act independently and autonomously. In the case of NSCLC, it was shown that the over-expression of this receptor, as well as specific somatic mutations occurred in their intracellular domain with tyrosine kinase activity (between exons 18 and 21), which may influence prognosis, being significantly related to stage, survival and chemotherapy response. These data led to the development and study of various substances, including monoclonal antibodies directed to the extracellular domain of EGFR (e.g., cetuximab, Erbitux®) and small molecules that inhibit the tyrosine kinase intracellular domain (tyrosine kinase inhibitors, TKIs) of EGFR (e.g., gefitinib and erlotinib). Preliminary results of randomized clinical trials conducted with these TKIs have shown that their use in patients with advanced disease is effective, significantly increasing the survival of these patients, especially if they harbor mutations in the EGFR which are more frequently found in a subgroup of non-smoking, female patients, of Asian ethnicity and with adenocarcinoma histological sub-type (especially in the presence of bronchioloalveolar carcinoma). Some of these results were so impressive that this phenomenon was designated, the Lazarus effect, and led to the approval, in the United States and Europe, of erlotinib for the second- and third-line treatment of NSCLC patients; and gefitinib in Europe, for patients harboring the EGFR mutation (del Mello, et al., World J Clinc Oncol, Vol. 2, p. 367 (2011)).

EGFR, also known as ErbB1 or Her1, is a transmembrane glycoprotein encoded by a gene located on chromosome 7 (7p12.1-12.3). EGFR comprises 1186 amino acids (a.a.) and 26 exons. Exons 1-14 encode the extracellular domain, exon 15 encodes the transmembrane region and exons 16-26 the intracellular domain. This glycoprotein belongs to the ErbB receptor family, which also consists of: ErbB2 (HER2/neu), ErbB3 (HER3) and ErbB4 (HER4). Each of these proteins is structurally composed of an extracellular domain, a hydrophobic transmembrane domain and an intracellular domain with intrinsic tyrosine kinase (TK) activity (except ErbB3). These receptors exist as inactive monomers, being activated by their interaction, through the extracellular domain, with growth factors of the EGF family. The binding of ErbB receptor molecules to one of these ligands leads to its interaction with other monomers of the same family (receptor dimerization). This dimerization can occur between two identical receptors (homodimerization, e.g., ErbB1-ErbB1) or between two different receptors (heterodimerization, e.g., ErbB1-ErbB3). The stimulation caused by a specific ligand triggers a unique pattern of dimerization, which is also specific to the tissue/tumor in which the phenomenon occurs. Dimerization of the receptors leads to their autophosphorylation with activation of TK and activation of a cascade of intracellular biochemical processes that regulate such diverse activities, like proliferation, differentiation, apoptosis and cell migration.

### E-cadherin

Epithelial cadherin (E-cadherin), also known as cadherin-1, CAM 120/80 or uvomorulin, is a protein that in humans is encoded by the CDH1 gene. E-cadherin is a classical member of the cadherin superfamily. E-cadherin is a calcium-dependent cell-cell adhesion glycoprotein composed of five extracellular cadherin repeats (EC1-EC5) in the extracellular domain, a transmembrane domain, an intracellular domain that binds p120-catenin and beta-catenin, and a highly conserved cytoplasmic tail. The intracellular domain contains a highly-phosphorylated region vital to beta-catenin binding and, therefore, to E-cadherin function. Beta-catenin can also bind to alpha-catenin Alpha-catenin participates in regulation of actin-containing cytoskeletal filaments. In epithelial cells, E-cadherin-containing cell-to-cell junctions are often adjacent to actin-containing filaments of the cytoskeleton.

Mutations in this gene are correlated with gastric, breast, colorectal, thyroid, and ovarian cancers. Loss of function or expression is thought to contribute to progression in cancer and metastasis. E-cadherin downregulation decreases the strength of cellular adhesion within a tissue, resulting in an increase in cellular motility. This in turn may allow cancer cells to cross the basement membrane and invade surrounding tissues.

### Methods for Determining E-Cadherin Levels

E-cadherin protein levels can be quantitatively measured by ELISA. Some E-cadherin ELISA kits, such as the E-cadherin EIA kit provided by TaKaRA, are a solid phase sandwich EIA that utilizes two mouse monoclonal E-cadherin antibodies (one of which is coated on the plate, and the other is POD-labeled) for detection of human E-cadherin using a two-step incubation method. In the first step, samples are incubated in the antibody-coated microtiter plate. During the second step, the plate is washed and incubated with the POD-labeled E-cadherin antibody. A substrate is added, and the reaction between POD and the substrate (H2O2, TMBZ) results in a color development. The amount of sample soluble E-cadherin is determined by measuring absorbance using an EIA plate reader. Accurate soluble E-cadherin sample concentrations can be determined by comparing their specific absorbances with the absorbance obtained for the Standard plotted on a standard curve. In some embodiments, E-cadherin protein levels are quantitatively measured by ELISA.

E-cadherin protein levels can be detected by immunohistochemistry. To detect E-cadherin levels in immersion fixed cells, cells are incubated with Human E-Cadherin Antigen Affinity-purified Polyclonal Antibody (R&D Systems® Catalog #AF648) at 10 μg/mL for 3 hours at room temperature. Cells are then stained using the NorthernLights™ 557-conjugated Anti-Goat IgG Secondary Antibody (R&D Systems® Catalog #NL001) and counterstained with DAPI. E-cadherin and DAPI can be visualized using a fluorescence microscope and filter sets appropriate for the label used. In some embodiments, E-cadherin protein levels are detected by immunhistochemistry.

E-cadherin protein levels can be detected by immunocytochemistry. Coverslips for immunocytochemistry (ICC) can be prepared using gelatin. In some embodiments, a method for preparing coverslips for ICC includes a) placing sterilized coverslips into the wells of a 24-well plate, b) adding 400 μL of the gelatin-coating solution and c) incubating the coverslips for 10 minutes at room temperature. Then the gelatin-coating solution is removed and the coverslips are air-dried for 15 minutes. The dried coverslips can be stored at room temperature until use. Once the coverslips have been prepared, the cells can be prepared and fixed as follows. Culture cells by adding 500 μL of culture media containing approximately 5000 cells to the wells of a cell culture plate containing gelatin-coated coverslips. When cells have reached the desired density/age, remove the culture media from each well and wash twice with PBS. Add 300-400 μL of 2-4% Formaldehyde Fixative Solution to each well, and incubate for 20 minutes at room temperature. Wash the wells twice with PBS and cover with 400 μL of wash buffer. The coverslips can be stored at 2-8° C. for up to 3 months or they may be stained immediately. Once the cells have been prepared, the cells can be stained for ICC as follows. Wash the coverslips containing the fixed cells two times in 400 μL of wash buffer. Block non-specific staining by adding 400 μL of blocking buffer and incubate for 45 minutes at room temperature. Remove blocking buffer. No rinsing is necessary. Dilute the unconjugated primary antibody (or fluorescence-conjugated primary) in dilution buffer according to the manufacturer's instructions. For fluorescent ICC staining of cells on coverslips using R&D Systems antibodies, it is recommended to incubate at room temperature for 1 hour. Alternatively, incubate overnight at 2-8° C. Wash two times in 400 μL of wash buffer. If using a primary antibody with a direct fluorescent conjugate, go to step 8. Dilute the secondary antibody in dilution buffer according to the manufacturer's instructions. Add 400 μL to the wells, and incubate at room temperature for 1 hour in the dark. From this step forward samples should be protected from light. Rinse two times in 400 μL of wash buffer. Add 300 μL of the diluted DAPI solution to each well, and incubate 2-5 minutes at room temperature. DAPI binds to DNA and is a convenient nuclear counterstain. It has an absorption maximum at 358 nm and fluoresces blue at an emission maximum of 461 nm. Rinse once with PBS and once with water. Carefully remove the coverslips from the wells and blot to remove any excess water. Dispense 1 drop of anti-fade mounting medium onto the microscope slide per coverslip. Mount the coverslip with the cells facing towards the microscope slide. Visualize using a fluorescence microscope and filter sets appropriate for the label used. Slides can also be stored in a slide box at <−20° C. for later examination. In some embodiments, E-cadherin protein levels are detected by immuncytochemistry.

E-cadherin gene expression can be determined by measuring E-cadherin methylation. E-cadherin methylation kits, such as the CpG WIZ® E-cadherin amplification kit provided by Millipore®, determine the methylation of status of the E-cadherin promoter by methylation-specific PCR (MSP). The kit contains primers targeted to regions of the promoter where the sequences are most divergent after bisulfate treatment. PCR parameters have been identified so that all primer sets in the kit amplify under the same conditions. Control genomic DNA samples (methylated and unmethylated) for E-cadherin are also included. In some embodiments, E-cadherin gene expression is determined by measuring E-cadherin methylation.

One embodiment provides a method of treating cancer in an EGFR inhibitor-naïve patient progressed on prior therapy, wherein said patient exhibits high E-cadherin expression levels, the method comprising administering to the patient a combination comprising entinostat and an EGFR inhibitor. Another embodiment provides the method wherein high E-cadherin expression levels are characterized by ELISA, immunohistochemistry, immunocytochemistry or determination of E-cadherin methylation levels. Another embodiment provides the method wherein high E-cadherin expression levels are determined by immunohistochemistry. Another embodiment provides the method wherein the high E-cadherin expression levels are scored as +3 as determined by immunohistochemistry.

### Lung Cancer

Lung cancer is the leading cause of cancer deaths in women and men both in the United States and throughout the world. Lung cancer has surpassed breast cancer as the leading cause of cancer deaths in women. In the United States in 2010, 157,300 people were projected to die from lung cancer, which is more than the number of deaths from colon and rectal, breast, and prostate cancer combined. Only about 2% of those diagnosed with lung cancer that has spread to other areas of the body are alive five years after the diagnosis, although the survival rates for lung cancers diagnosed at the earliest stage are higher, with approximately 49% surviving for five years or longer.

Cancer occurs when normal cells undergo a transformation that causes them to grow and multiply without control. The cells form a mass or tumor that differs from the surrounding tissues from which it arises. Tumors are dangerous because they take oxygen, nutrients, and space from healthy cells and because they invade and destroy or reduce the ability of normal tissues to function.

Most lung tumors are malignant. This means that they invade and destroy the healthy tissues around them and can spread throughout the body. The tumors can spread to nearby lymph nodes or through the bloodstream to other organs. This process is called metastasis. When lung cancer metastasizes, the tumor in the lung is called the primary tumor, and the tumors in other parts of the body are called secondary tumors or metastatic tumors.

Some tumors in the lung are metastatic from cancers elsewhere in the body. The lungs are a common site for metastasis. If this is the case, the cancer is not considered to be lung cancer. For example, if prostate cancer spreads via the bloodstream to the lungs, it is metastatic prostate cancer (a secondary cancer) in the lung and is not called lung cancer.

Lung cancer comprises a group of different types of tumors. Lung cancers usually are divided into two main groups that account for about 95% of all cases. The division into groups is based on the type of cells that make up the cancer. The two main types of lung cancer are characterized by the cell size of the tumor when viewed under the microscope. They are called small cell lung cancer (SCLC) and non-small cell lung cancer (NSCLC). NSCLC includes several subtypes of tumors. SCLCs are less common, but they grow more quickly and are more likely to metastasize than NSCLCs. Often, SCLCs have already spread to other parts of the body when the cancer is diagnosed. About 5% of lung cancers are of rare cell types, including carcinoid tumor, lymphoma, and others. As used herein, the term “lung cancer” includes, but is not limited to, SCLC, NSCLC, carcinoid tumor, lymphoma, and their various subtypes.

### Non-Small Cell Lung Cancer

NSCLC is a cancer of the lung which is not of the small cell carcinoma (oat cell carcinoma) type. The term “non-small cell lung cancer” applies to the various types of bronchogenic carcinomas (those arising from the lining of the bronchi). Examples of specific types of NSCLC include, but are not limited to, adenocarcinoma, squamous cell carcinoma, and large cell cancer (i.e., large cell undifferentiated carcinoma).

Adenocarcinoma is a cancer that develops in the lining or inner surface of an organ. Adenocarcinoma is the most common type of lung cancer, making up 30%-40% of all cases of lung cancer. A subtype of adenocarcinoma is called bronchoalveolar cell carcinoma, which creates a pneumonia-like appearance on chest X-rays.

Squamous cell carcinoma is a cancer that begins in squamous cells. Squamos cells are thin, flat cells that look under the microscope like fish scales. Squamous cells are found in the tissue that forms the surface of the skin, the lining of hollow organs of the body, and the passages of the respiratory and digestive tracts. Squamous cell carcinomas may arise in any of these tissues. Squamous cell carcinoma is the second most common type of lung cancer, making up about 30% of all cases.

Large cell carcinoma shows no evidence of squamous or glandular maturation. Thus these tumors are often diagnosed by default, when all other possibilities have been excluded. These tumors lack any diagnostic features to suggest their diagnosis prior to biopsy. They tend to grow rapidly, metastasize early, and are strongly associated with smoking. Large cell tumors are usually large, bulky, well-circumscribed, pink-grey masses with extensive hemorrhage and necrosis. Although they commonly have central necrosis, they rarely cavitate. They tend to present in the mid to peripheral lung zones. They may extend locally to involve the segmental or subsegmental bronchi. A variant of large cell carcinoma is giant cell carcinoma. This subtype is particularly aggressive and carries a very poor prognosis. These tumors generally present as a large peripheral mass with a focal necrotic component. They do not involve the large airways, unless by direct extension. Large cell cancer makes up 10%-20% of all cases of lung cancer.

### Small Cell Lung Cancer

SCLC is also called oat cell lung cancer and is a type of lung cancer in which the cells appear small and round under the microscope. SCLC is considered distinct from other lung cancers because of their clinical and biologic characteristics. Small cell lung cancer exhibits aggressive behavior, with rapid growth, early spread to distant sites, exquisite sensitivity to chemotherapy and radiation, and frequent association with distinct paraneoplastic syndromes. Small cell carcinomas arise in peribronchial locations and infiltrate the bronchial submucosa. Widespread metastases occur early in the course of the disease, with common spread to the mediastinal lymph nodes, liver, bones, adrenal glands, and brain. In addition, production of various peptide hormones leads to a wide range of paraneoplastic syndromes; the most common of these is the syndrome of inappropriate secretion of antidiuretic hormone (SIADH) and the syndrome of ectopic adrenocorticotropic hormone (ACTH) production. In addition, autoimmune phenomena may lead to various neurologic syndromes, such as Lambert-Eaton syndrome. SCLC makes up 20% of all cases.

### Carcinoid Tumor

Carcinoid tumor is a tumor which secretes large amounts of the hormone serotonin Carcinoid tumor is also called an argentaffinoma. The tumor usually arises in the gastrointestinal tract, anywhere between the stomach and the rectum (the favorite spot is in the appendix) and from there may metastasize to the liver. In the liver the tumor produces and releases large quantities of serotonin into the systemic bloodstream. The consequences are called the carcinoid syndrome. It is directly due to the serotonin and includes flushing and blushing, swelling of the face (especially around the eyes), flat angiomas (little collections of dilated blood vessels) on the skin, diarrhea, bronchial spasm, rapid pulse, low blood pressure and tricuspid and pulmonary stenosis (narrowing of the tricuspid and pulmonic valves of the heart), often with regurgitation. One or more of four kinds of treatment are used for carcinoid tumors: surgery (to take out the cancer); radiation therapy (using high-dose x-rays to kill the cancer cells); biological therapy (using the body's natural immune system to fight the cancer); and chemotherapy (using drugs to kill cancer cells). Carcinoid tumors are considered a type of endocrine tumor since they secrete a hormone (serotonin). They can occur as part of certain genetic disorders such as the multiple endocrine neoplasia (MEN) type 1 and neurofibromatosis type 1 (NF1 or von Recklinghausen disease). Carcinoid tumors account for 1% of all cases.

### Lymphoma

Lymphoma is a type of cancer involving cells of the immune system, called lymphocytes, and primarily represents cells involved in the lymphatic system of the body. Lymphoma is a malignant transformation of either B or T cells or their subtypes. Lymphomas fall into one of two major categories: Hodgkin's lymphoma (HL, previously called Hodgkin's disease) and all other lymphomas (non-Hodgkin's lymphomas or NHLs). These two types occur in the same places, may be associated with the same symptoms, and often have similar appearance on physical examination. However, they are readily distinguishable via microscopic examination. Hodgkin's disease develops from a specific abnormal B lymphocyte lineage. NHL may derive from either abnormal B or T cells and are distinguished by unique genetic markers. There are five subtypes of Hodgkin's disease and about 30 subtypes of non-Hodgkin's lymphoma. Because there are so many different subtypes of lymphoma, the classification of lymphomas is complicated (it includes both the microscopic appearance as well as genetic and molecular markers). Many of the NHL subtypes look similar, but they are functionally quite different and respond to different therapies with different probabilities of cure. HL subtypes are microscopically distinct, and typing is based upon the microscopic differences as well as extent of disease.

### HDAC Inhibitors

HDAC inhibitors can be classified broadly into pan HDAC inhibitors and selective HDAC inhibitors. Although there is a large structural diversity of known HDAC inhibitors, they share common features: a part that interacts with the enzyme active site and a side-chain that sits inside the channel leading to the active site. This can be seen with the hydroxamates such as SAHA, where the hydroxamate group is believed to interact with the active site. In the case of the depsipeptides, it is believed that an intracellular reduction of the disulphide bond creates a free thiol group (which interacts with the active site) attached to a 4-carbon alkenyl chain. A difference between the HDAC inhibitors is in the way that they interact with the rim of the HDAC channel, which is at the opposite end of the channel to the active site. It is this interaction, between the HDAC inhibitor and the rim of the channel, which is believed to account, at least in part, for some observed differences in HDAC selectivity between pan-HDAC inhibitors, such as SAHA and selective HDAC inhibitors such as the depsipeptides. A particularly preferred HDAC inhibitor is entinostat. Entinostat has the chemical name N-(2-aminophenyl)-4-[N-(pyridine-3-yl)methoxycarbonylamino-methyl]-benzamide and the chemical structure shown below.

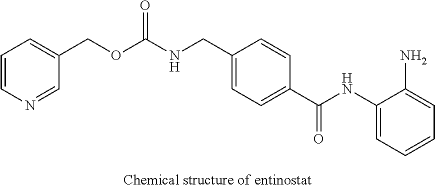 [SMILES: Nc1ccccc1NC(=O)c1ccc(CNC(=O)OCc2cccnc2)cc1]

EGFR Inhibitors

EGFR inhibitors interrupt signaling through the epiderdermal growth factor receptor (EGFR) in target cells. Certain EGFR inhibitors, such as erlotinib, have been approved for the treatment of metastatic NSCLC. For advanced NSCLC, EGFR inhibitors, such as gefitinib, have been approved. Several more EGFR inhibitors are being tested in clinical trials for the treatment of NSCLC and additional lung cancers.

As described herein, an “EGFR inhibitor” is a molecule which inhibits the activity of the EGF receptor. Compounds which are inhibitors of EGFR can be readily identified by one skilled in the art using methods such as, for example, an EGFR kinase assay which measures ADP formed from a kinase reaction.

Inhibition of EGFR as a treatment option for lung cancer has been studied with some success. Currently three EGFR inhibitors, erlotinib, gefinitib, and cetuximab, are approved for marketing in the US for the treatment of lung cancer. Erlotinib (Tarceva®) is approved to treat metastatic non-small cell lung cancer and pancreatic cancer that cannot be removed by surgery or has metastasized. This small-molecule drug inhibits the tyrosine kinase activity of EGFR.

Gefitinib (Iressa®) is approved to treat patients with advanced non-small cell lung cancer. This small-molecule drug is restricted to use in patients who, in the opinion of their treating physician, are currently benefiting, or have previously benefited, from gefitinib treatment. Gefitinib inhibits the tyrosine kinase activity of the epidermal growth factor receptor (EGFR), which is overproduced by many types of cancer cells.

Cetuximab (Erbitux®) is a monoclonal antibody that is approved for treating some patients with squamous cell carcinoma of the head and neck or colorectal cancer. The therapy binds to the external portion of EGFR, thereby preventing the receptor from being activated by growth signals, which may inhibit signal transduction and lead to antiproliferative effects.

Additional examples of EGFR inhibitors include, but are not limited to, panitumumab, vandetanib, lapatinib, canertinib, afatinib, necitumumab, nimotuzumab, PF299804, RO5083945, ABT-806, and AP26113.

Panitumumab (Vectibix®) is approved to treat some patients with metastatic colon cancer. This monoclonal antibody attaches to EGFR and prevents it from sending growth signals.

Vandetanib (Caprelsa®) is approved to treat patients with metastatic medullary thyroid cancer who are ineligible for surgery. This small-molecule drug binds to and blocks the growth-promoting activity of several tyrosine kinase enzymes, including EGFR, several receptors for vascular endothelial growth factor receptor (VEGF), and RET.

Lapatinib (Tykerb®) is approved for the treatment of certain types of advanced or metastatic breast cancer. This small-molecule drug inhibits several tyrosine kinases, including the tyrosine kinase activity of HER-2. Lapatinib treatment prevents HER-2 signals from activating cell growth.

Canertinib is an orally bioavailable irreversible pan-ErbB tyrosine kinase inhibitor, targeting EGFR, HER-2, ErbB-3 and ErbB-4. It effectively inhibits the growth of esophageal squamous cell carcinoma which co-expresses both EGFR and HER2 with the inhibition of phosphorylation of both MAPK and AKT. In vitro studies of human cancer cell lines indicate that canertinib results in prompt, potent, and sustained inhibition of tyrosine kinase activity.

Afatinib is an irreversible EGFR/HER2. In cell-free in vitro kinase assays, afatinib shows potent activity against wild-type and mutant forms of EGFR and HER2, similar to gefitinib in potency for L858R EGFR, but about 100-fold more active against the gefitinib resistant L858R-T790M EGFR double mutant. Afatinib was effective in inhibiting survival of lung cancer cell lines harboring wild-type (H1666) or L858R/T790M (NCI-H1975) EGFR. Assessed in a standard xenograft model of the epidermoid carcinoma cell line A431. Daily oral treatment with afatinib at 20 mg/kg for 25 days resulted in dramatic tumor regression with a cumulative treated/control tumor volume ratio (T/C ratio) of 2%. Like lapatinib and neratinib, afatinib is a next generation tyrosine kinase inhibitor (TKI) that irreversibly inhibits human epidermal growth factor receptor 2 (Her2) and epidermal growth factor receptor (EGFR) kinases. Afatinib is not only active against EGFR mutations targeted by first generation TKIs like erlotinib or gefitinib, but also against those not sensitive to these standard therapies. Because of its additional activity against Her2, it is investigated for breast cancer as well as other EGFR and Her2 driven cancers.

Necitumumab is a fully human IgG1 monoclonal antibody directed against the epidermal growth factor receptor (EGFR) with potential antineoplastic activity. Necitumumab binds to and blocks the ligand binding site of EGFR, thereby preventing the activation and subsequent dimerization of the receptor. This may lead to an inhibition of EGFR-dependent downstream pathways and so inhibition of EGFR-dependent tumor cell proliferation and metastasis.

Nimotuzumab is a humanized monoclonal antibody directed against the epidermal growth factor receptor (EGFR) with potential antineoplastic activity. Nimotuzumab binds to and inhibits EGFR, resulting in growth inhibition of tumor cells that overexpress EGFR. This agent may act synergistically with radiation therapy.

PF299804 is a potent, irreversible inhibitor of human epidermal growth factor receptor (HER)-1/EGFR, -2, and -4 tyrosine kinases (TK), is active in E-sensitive and -resistant preclinical models. PF299804 had clinical activity in phase I/II trials in EGFR TK inhibitor (TKI)-refractory NSCLC.

RO5083945 is a glycoengineered anti EGFR IgG1 mAb exhibiting increased binding affinity for all FcγRIIIa variants expressed on immune effector cells. RO5083945 demonstrates significantly improved cell killing in ADCC-based assays and greater activity in in vivo models compared to cetuximab and panitumumab. Hence, RO5083945 has the potential to show clinical activity in patients with solid tumors, including KRAS mutant CRC.

ABT-806 is a humanized monoclonal antibody (MoAb) against human epidermal growth factor receptor (EGFR) with antineoplastic activity. MoAb ABT-806 targets the EGFR deletion variant, de2-7 EGFR as well as wild-type EGFR expressed in cells overexpressing the receptor, thereby preventing the activation and subsequent dimerization of the receptor; the decrease in receptor activation and dimerization result in an inhibition in signal transduction and anti-proliferative effects. This MoAb targets cells expressing aberrant EGFR, hence making it an ideal candidate for generation of radioisotope or toxin conjugates.

AP26113 is an orally available inhibitor of receptor tyrosine kinases anaplastic lymphoma kinase (ALK) and the epidermal growth factor receptor (EGFR) with potential antineoplastic activity. Dual ALK/EGFR inhibitor AP26113 binds to and inhibits ALK kinase and ALK fusion proteins as well as EGFR and mutant forms. This leads to the inhibition of ALK kinase and EGFR kinase, disrupts their signaling pathways and eventually inhibits tumor cell growth in susceptible tumor cells. In addition, AP26113 appears to overcome mutation-based resistance. ALK belongs to the insulin receptor superfamily and plays an important role in nervous system development; ALK dysregulation and gene rearrangements are associated with a series of tumors. EGFR is overexpressed in a variety of cancer cell types.

### Additional Therapy

Available additional treatments for lung cancer that may be advantageously employed in combination with the therapies disclosed herein include, without limitation, radiation therapy, chemotherapy, antibody therapy, and tyrosine kinase inhibitors as adjuvant therapy.

Radiation therapy is a cancer treatment that uses high-energy x-rays or other types of radiation to kill cancer cells or keep them from growing. Chemotherapy is a cancer treatment that uses drugs to stop the growth of cancer cells, either by killing the cells or by stopping them from dividing. When chemotherapy is taken by mouth or injected into a vein or muscle, the drugs enter the bloodstream and can reach cancer cells throughout the body (systemic chemotherapy). When chemotherapy is placed directly into the spinal column, an organ, or a body cavity such as the abdomen, the drugs mainly affect cancer cells in those areas (regional chemotherapy). The way the chemotherapy is given depends on the type and stage of the cancer being treated.

Different chemotherapeutic agents are known in the art for treating lung cancer. Cytoxic agents used for treating lung cancer include carboplatin (for example, Paraplatin®, Paraplat®), cisplatin (for example, Platinol®, Platinol-Aq®), crizotinib (for example Xalkori®), etoposide (for example Toposar®, VePesid®), etoposide Phosphate (for example Etopophos®), gemcitabine hydrochloride (for example Gemzar®), gemcitabine-cisplatin, methotrexate (for example Abitrexate®, Folex®, Folex Pfs®, Methotrexate Lpf®, Mexate®, Mexate-Aq®), paclitaxel (for example Taxol®), pemetrexed Disodium (for example Alimta®), and topotecan Hydrochloride (for example Hycamtin®)

Monoclonal antibody therapy is a cancer treatment that uses antibodies made in the laboratory, from a single type of immune system cell. These antibodies can identify substances on cancer cells or normal substances that may help cancer cells grow. The antibodies attach to the substances and kill the cancer cells, block their growth, or keep them from spreading. Monoclonal antibodies are given by infusion. They may be used alone or to carry drugs, toxins, or radioactive material directly to cancer cells. Monoclonal antibodies are also used in combination with chemotherapy as adjuvant therapy.

Bevacizumab (Avastin®) is a recombinant humanized monoclonal antibody directed against the vascular endothelial growth factor (VEGF), a pro-angiogenic cytokine. Bevacizumab binds to VEGF and inhibits VEGF receptor binding, thereby preventing the growth and maintenance of tumor blood vessels. Bevacizumab is used currently to treat several types of cancer, including certain types of colorectal, lung, breast, and kidney cancers and glioblastoma.

Additional, illustrative, treatments that may be advantageously combined with the compositions and therapies disclosed herein may include, without limitation, administration of agents including, but not limited to lapatinib, alone or in combination with capecitabine, docetaxel, epirubicin, epothilone A, B or D, goserelin acetate, paclitaxel, pamidronate, bevacizumab, or trastuzumab.

In some embodiments, the additional therapy comprises chemotherapy comprising administering to the subject one or more of doxorubicin, cyclophosphamide, paclitaxel, lapatinib, capecitabine, trastuzumab, bevacizumab, gemcitabine, eribulin, or nab-paclitaxel.

### Oral Formulations

Oral formulations containing the active pharmaceutical ingredients described herein may comprise any conventionally used oral forms, including: tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, syrups, buccal forms, and oral liquids. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. In some embodiments are surface modifying agents which include nonionic and anionic surface modifying agents. For example, surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine Oral formulations herein may utilize standard delay or time release formulations to alter the absorption of the active compound(s). The oral formulation may also consist of administering the active ingredient in water or a fruit juice, containing appropriate solubilizers or emulsifiers as needed.

### Oral Administration

As described herein, the combination therapy described herein can be given simultaneously or can be given in a staggered regimen, with entinostat being given at a different time during the course of chemotherapy than the EGFR inhibitor. This time differential may range from several minutes, hours, days, weeks, or longer between administrations of the two compounds. Therefore, the term combination does not necessarily mean administered at the same time or as a unitary dose, but that each of the components are administered during a desired treatment period. The agents may also be administered by different routes. As is typical for chemotherapeutic regimens, a course of chemotherapy may be repeated several weeks later, and may follow the same timeframe for administration of the two compounds, or may be modified based on patient response.

In other embodiments, the pharmaceutical compositions provided herein may be provided in solid, semisolid, or liquid dosage forms for oral administration. As used herein, oral administration also include buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include colloidal silicon dioxide, CAB-O-SIL® (Cabot Co. of Boston, Mass.), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many carriers and excipients may serve several functions, even within the same formulation.

In further embodiments, the pharmaceutical compositions provided herein may be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein may be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

In other embodiments, the pharmaceutical compositions provided herein may be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration may be also provided in the forms of liposomes, micelles, microspheres, or nanosystems. Miccellar dosage forms can be prepared as described in U.S. Pat. No. 6,350,458.

In other embodiments, the pharmaceutical compositions provided herein may be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions provided herein may be formulated as immediate or modified release dosage forms, including delayed-, sustained, pulsed-, controlled, targeted-, and programmed-release forms.

In further embodiments, the pharmaceutical compositions provided herein may be co-formulated with other active ingredients which do not impair the desired therapeutic action, or with substances that supplement the desired action.

### EXAMPLES

### Example 1

A Phase 2 Exploratory Study of Erlotinib and Entinostat in Patients with Non-Small Cell Lung Carcinoma Who are Progressing on Erlotinib

The combination of entinostat with erlotinib in patients who are progressing on erlotinib will show measurable activity as evidenced by the disease control rate and with an acceptable safety profile.

Primary Outcome Measures:

Disease control rate (complete response, partial response, or stable disease for at least 3 months)

Secondary Outcome Measures:

Progression-free survival rate at 2 months

Progression-free survival rate at 4 months

Study Design

| Arm | Assigned Interventions |
|---|---|
| 1: Experimental | Drug: entinostat |
| “Erlotinib-responsive” patients are | entinostat (10 mg fixed dose PO |
| those who progressed following | Q2W) on days 1 and 15 of a 28-day |
| either a complete or partial | cycle for up to 6 cycles |
| response to erlotinib or a | Drug: erlotinib |
| period of stable disease | erlotinib (150 mg PO QD) for up to |
| lasting at least 3 months. | six (6) 28-day cycles |
| Interventions: | |
| Drug: entinostat | |
| Drug: erlotinib | |
| 2: Experimental | Drug: entinostat |
| “Erlotinib-nonresponsive” patients | entinostat (10 mg fixed dose PO |
| are those who either progressed | Q2W) on days 1 and 15 of a 28-day |
| immediately during treatment with | cycle for up to 6 cycles |
| erlotinib (i.e. after at least 1 full | Drug: erlotinib |
| cycle of erlotinib treatment) or | erlotinib (150 mg PO QD) for up to |
| had an objective response or period | six (6) 28-day cycles |
| of stable disease lasting less than | |
| 3 months. | |
| Interventions: | |
| Drug: entinostat | |
| Drug: erlotinib | |

### Eligibility Criteria

Ages Eligible for Study: 18 Years and older

### Genders Eligible for Study: Both

### Accepts Healthy Volunteers: No

### Inclusion Criteria:

Cytologically or histologically confirmed NSCLC of stage Mb (pleural effusion) or IV
Disease is progressing (either no response to treatment or subsequent relapse after an objective response) on erlotinib treatment, based on at least 2 scans (the last being within 4 weeks of study enrollment and can serve as the baseline scan for the patient's screening into the study)
Recovered from any toxicity associated with the most recent cancer treatment (no greater than grade 1 toxicity on CTCAE scale or to prior baseline condition)
At least 1 measurable lesion≧20 mm by conventional CT scan or ≧10 mm by spiral CT scan
ECOG performance score of 0, 1, or 2 and life expectancy of at least 3 months
Paraffin-embedded tumor specimen available for correlative studies
Male or female over 18 years of age
Hemoglobin≧9.0 g/dL; platelets≧75×10⁹/L; ANC≧1.0×10⁹/L without the use of hematopoietic growth factors
Coagulation tests within the normal range
Bilirubin and creatinine less than 2 times the upper limit of normal for the institution
AST and ALT less than 3 times the upper limit of normal for the institution
Potassium, magnesium and phosphorus within the normal range for the institution (supplementation is permissible)
Willing to use accepted and effective methods of contraception during the study (both men and women as appropriate) and for 3 months after the last dose of SNDX-275
Patient or legally acceptable representative has granted written informed consent before any study-specific procedure (including special screening tests) is performed

### Exclusion Criteria:

Prior stem cell transplant
Symptomatic CNS involvement
Prior treatment with an HDAC inhibitor
Concurrent anticancer therapy, with the exception of radiotherapy for a non-target study lesion
Currently taking medication(s) on the prohibited medication list
Systemic chemotherapy or treatment with an investigational agent within 28 days before enrollment
Current use of valproic acid
Untreated or unstable brain metastases, or taken steroids for this condition within 4 weeks of study drug administration
Currently active second malignancy, or any malignancy within the last 5 years other than cured basal or squamous cell skin carcinoma, cervical carcinoma in situ, or superficial bladder cancer
Inability to swallow oral medications or a gastrointestinal malabsorption condition
Uncontrolled infection requiring IV antibiotics, antivirals, or antifungals, known HIV infection, or active hepatitis B or C infection
Abnormal cardiac function as defined as clinically significant findings on ECG (multifocal PVCs, ST-T wave changes consistent with myocardial infarction or acute ischemia, QTc greater than 500 milliseconds), tachycardia, or left ventricular ejection fraction less than 40% on MUGA scan
Another serious or uncontrolled medical condition within 3 months of enrollment such as hypertension, diabetes mellitus, or suppressed immune system
Known hypersensitivity to benzamides
Morbid obesity
Women who are currently pregnant or breast-feeding
Patient is currently enrolled in (or completed within 28 days) another investigational drug study
Patient unavailable for on-study or follow-up assessments
Patient has any kind of medical, psychiatric, or behavioral disorder that places the patient at increased risk for study participation or compromises the ability of the patient to give written informed consent and/or to comply with study procedures and requirement

### CONCLUSIONS

In the clinical study disclosed herein comparable outcomes were observed with the entinostat/erlotinib combination and erlotinib/placebo in unselected patients (N=132). A median progression-free survival difference of 0.4 months and median overall survival difference of 2.2 months was not statistically significant.

Analysis of the clinical study results for outcome based on E-cadherin biomarker status indicated the overall survival of the E-cad 3+ sub-group [N=26] of the entinostat/erlotinib arm was 9.4 months vs. 5.4 months in the erlotinib/placebo group (hazard ratio 0.36; P=0.03).

The entinostat/erlotinib combination therapy was tolerable with no unexpected adverse events and a manageable safety profile. This result indicates a subpopulation of NSCLC patients having high e-cadherin expression levels for which entinostat provides the ability to overcome erlotinib resistance

The provided figures provide additional details of the clinical study described herein:

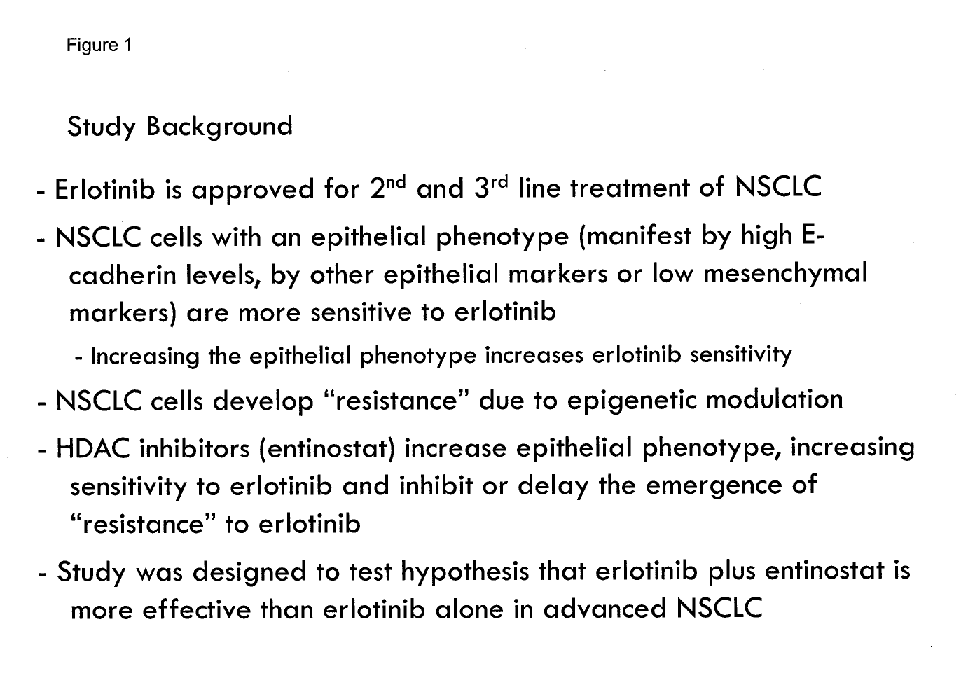

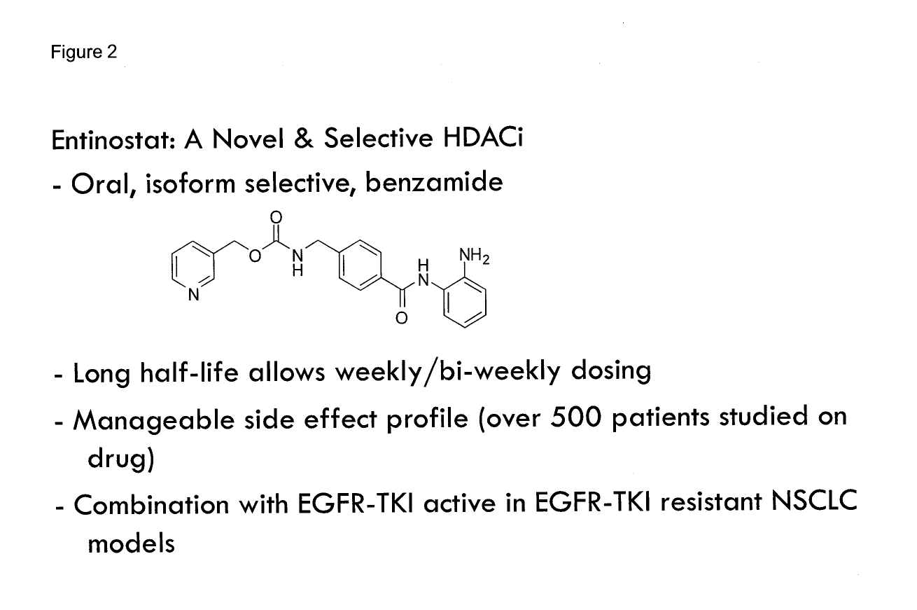

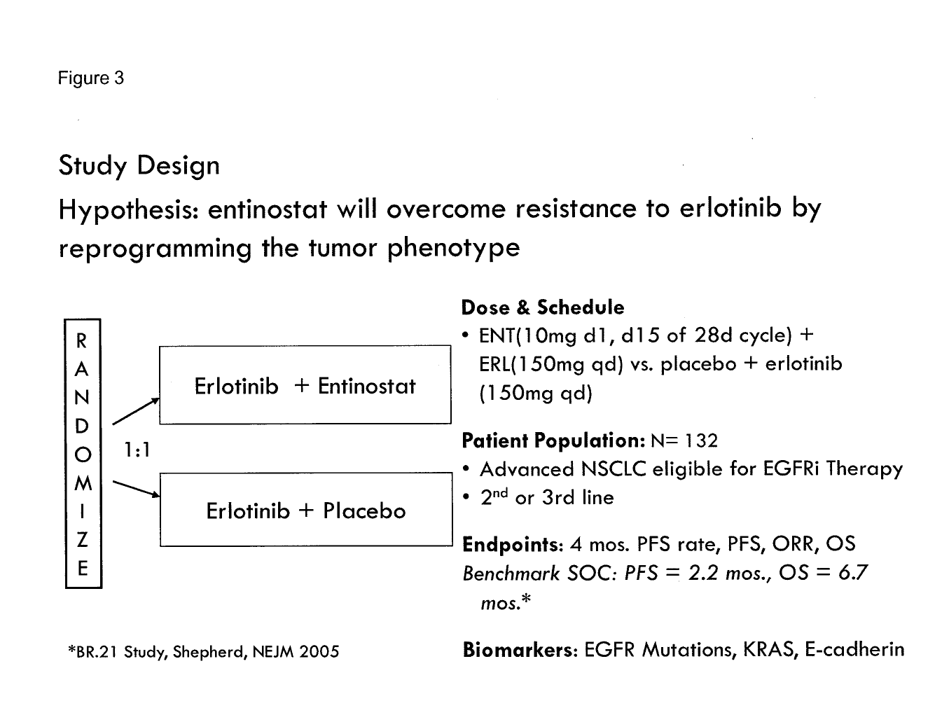

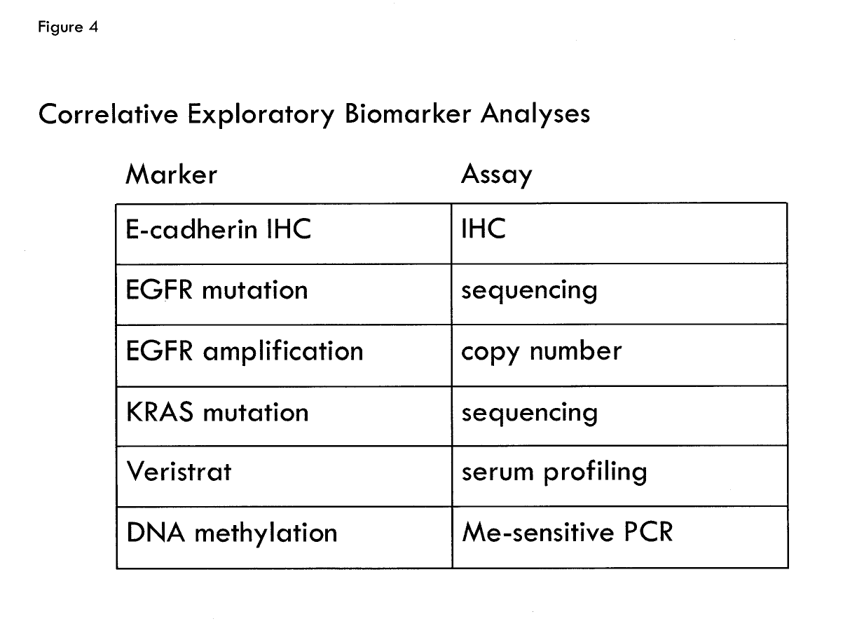

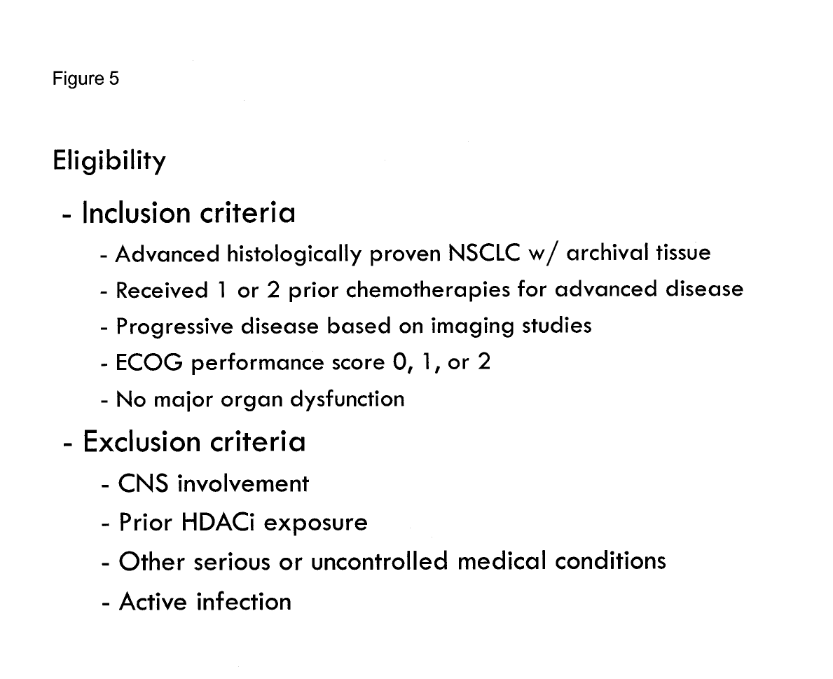

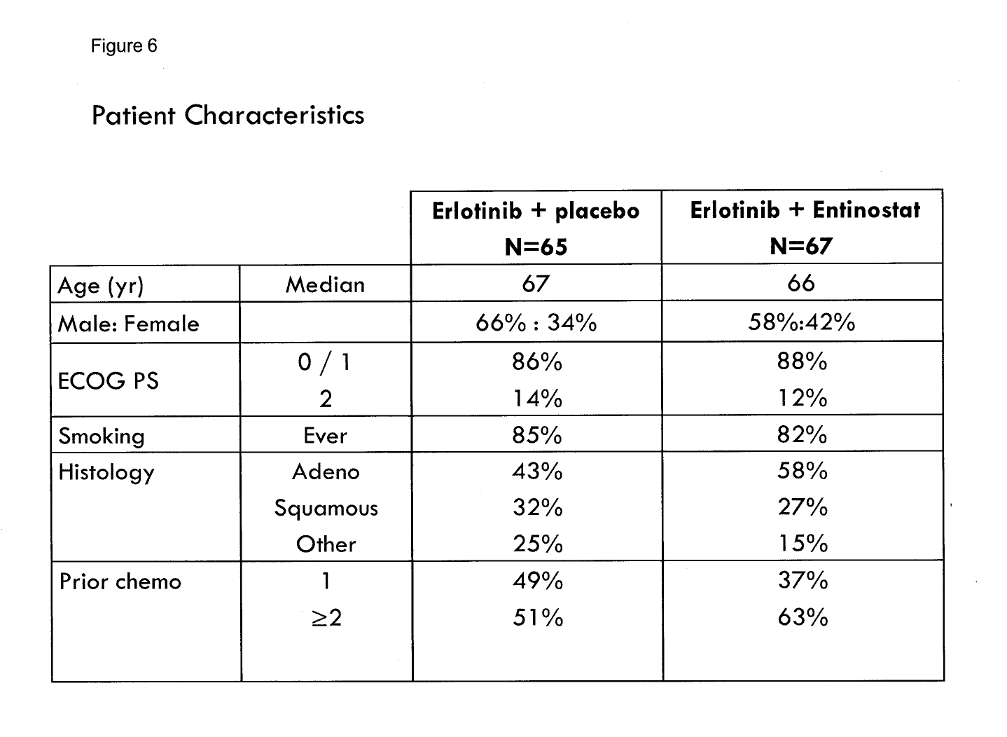

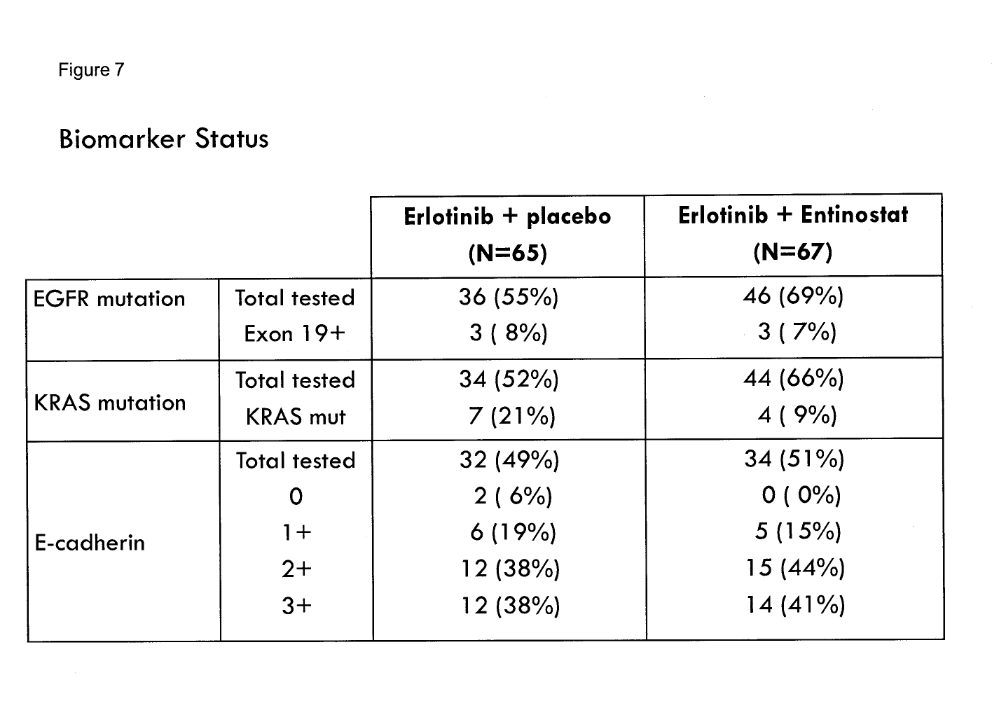

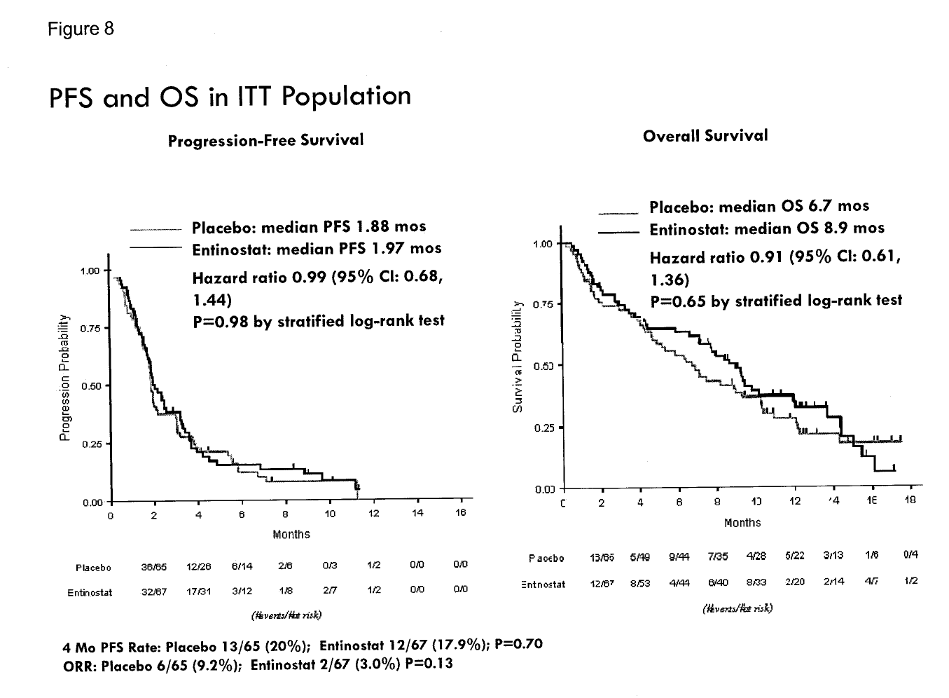

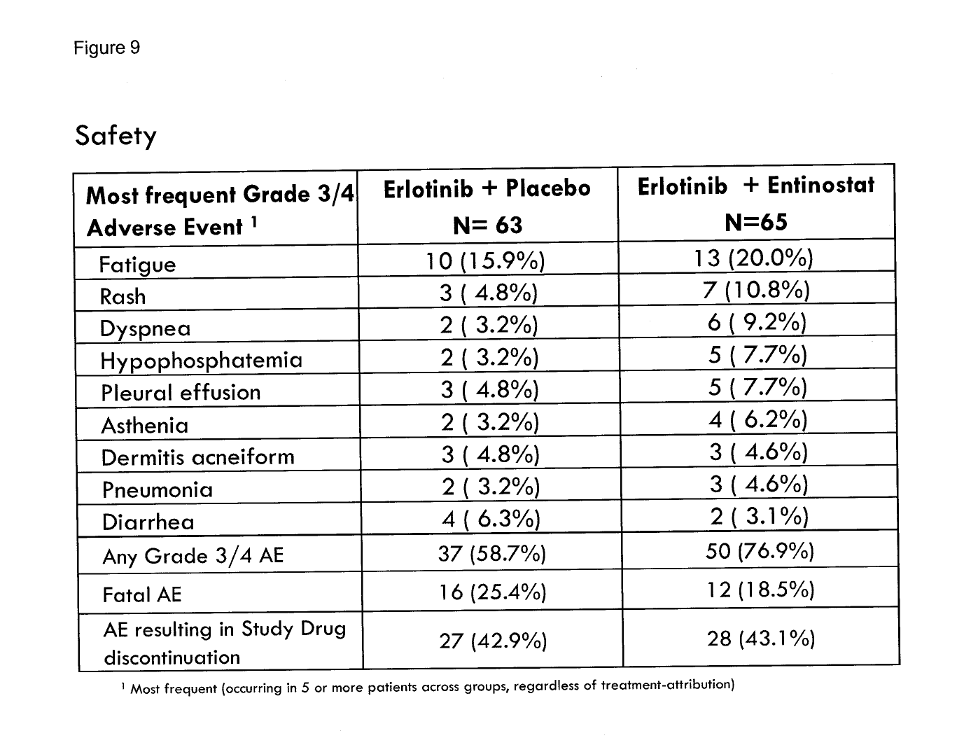

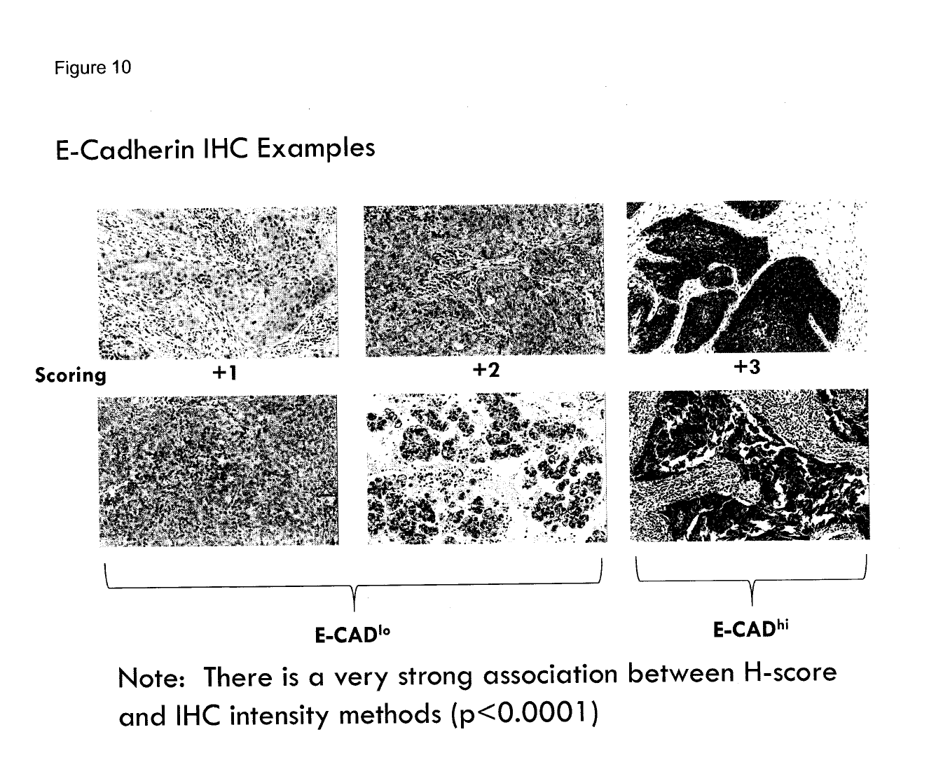

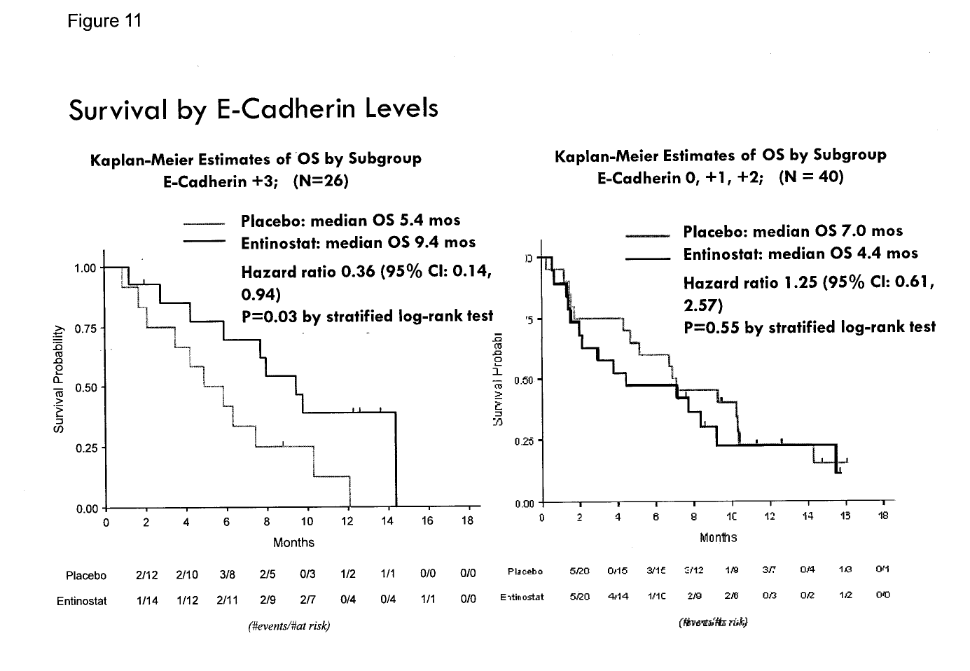

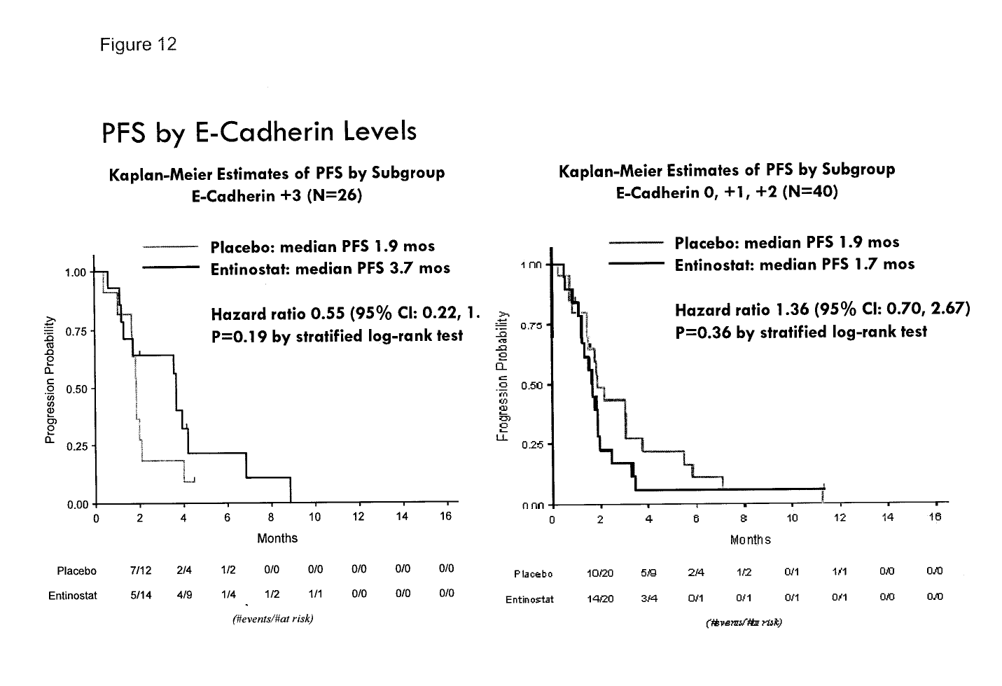

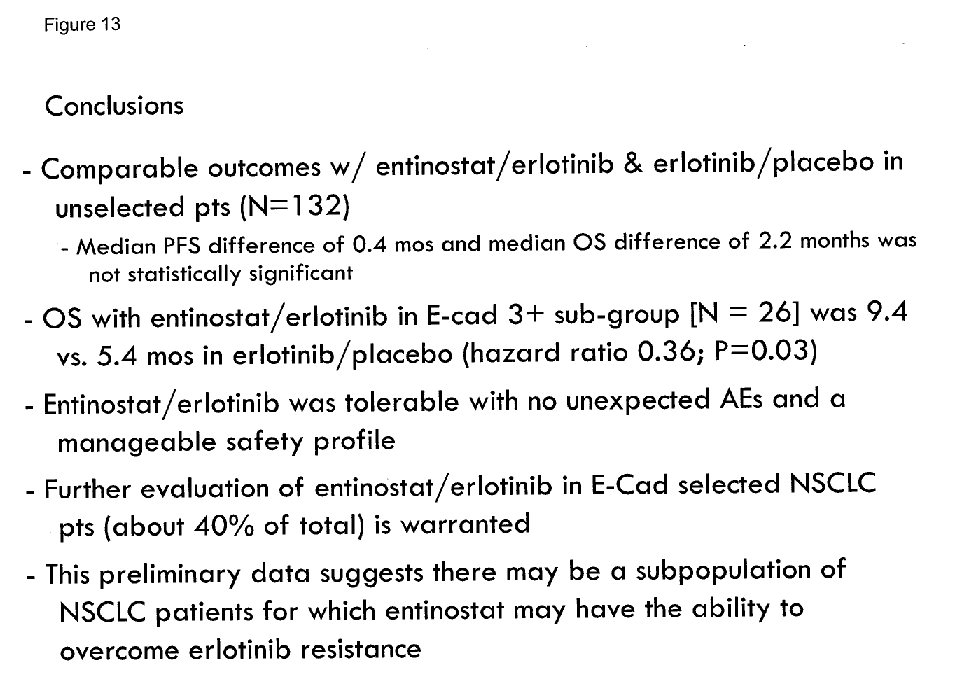

FIG. 1 provides a summary of the Phase II clinical trial;
FIG. 2 provides a summary of entinostat properties;
FIG. 3 provides an summary of the study design in the Phase II clinical trial;
FIG. 4 provides a summary of biomarkers and methods for analysis;
FIG. 5 provides a summary eligibility criteria for participation in the study;
FIG. 6 provides a summary of the patient population participating in the study described herein;
FIG. 7 provides a summary of biomarker status of the patient population participating in the study described herein;
FIG. 8 provides a summary of progression-free survival and overall survival observed during the Phase II clinical trial;
FIG. 9 provides a summary of adverse events observed during in the Phase II clinical trial;
FIG. 10 provides examples of E-cadherin protein level expression classification based on IHC analysis;
FIG. 11 provides an analysis of overall survival based upon E-cadherin levels;
FIG. 12 provides an analysis of progression-free survival based upon E-cadherin levels; and
FIG. 13 provides a summary of the outcome of the Phase II clinical trial.

What is claimed is:

1. A method of treating cancer in an EGFR inhibitor-naïve patient progressed on prior therapy, wherein the method comprises:
(1) determining the E-cadherin expression level in the patient;
(2) selecting the patient exhibiting a high E-cadherin expression level scored as +3; and
(3) administering to the patient a combination comprising entinostat and an EGFR inhibitor.

2. The method of claim 1, wherein the prior therapy was one prior chemotherapy.

3. The method of claim 1, wherein the prior therapy was two or more prior chemotherapies.

4. The method of claim 1, wherein high E-cadherin expression levels are determined by ELISA, immunohistochemistry, immunocytochemistry or determination of E-cadherin methylation levels.

5. The method of claim 1, wherein high E-cadherin expression levels are determined by immunohistochemistry.

6. The method of claim 1, wherein the cancer is lung cancer.

7. The method of claim 6, wherein the lung cancer is non-small cell lung cancer.

8. The method of claim 1, wherein the EGFR inhibitor administered in combination with entinostat is erlotinib.

9. The method of claim 1, wherein entinostat and the EGFR inhibitor are administered sequentially in either order or simultaneously.

10. The method of claim 1, wherein entinostat and the EGFR inhibitor are administered simultaneously.

11. The method of claim 1, wherein entinostat is administered first.

12. The method of claim 1, wherein the EGFR inhibitor is administered daily and the entinostat is administered periodically.

13. The method of claim 1, wherein the EGFR inhibitor is administered daily and the entinostat is administered weekly.

14. A kit for treating advanced non-small cell lung cancer comprising a combination of entinostat and an EGFR inhibitor and instructions for the administration of the dosage form.

15. The kit of claim 14, wherein the kit comprises one entinostat dosage form for every seven EGFR inhibitor dosage forms.

16. The kit of claim 14, wherein the kit comprises two entinostat dosage forms for every 14 EGFR inhibitor dosage forms.

17. The kit of claim 14, wherein the kit comprises four entinostat dosage forms and 28 EGFR inhibitor dosage forms.

18. The kit of claim 14, wherein the EGFR inhibitor is erlotinib.
